(19) United States
(12) Patent Application Publication
(10) Pub. No.: US 2007/0049523 A1
(43) Pub. Date: Mar. 1, 2007

(54) Liquid composition of modified factor VII polypeptides

(75) Inventors: Birthe Lykkegaard Hansen, Vaerlose (DK); Michael Bech Jensen, Allerod (DK); Troels Kornfelt, Virum (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/473,387

(22) Filed: Jun. 21, 2006

Related U.S. Application Data
(63) Continuation of application No. 10/602,340, filed on Jun. 23, 2003, now abandoned.

(30) Foreign Application Priority Data
Dec. 21, 2001 (DK) PA 2001 01948
Dec. 21, 2001 (DK) PA 2001 01949
Dec. 20, 2002 (WO) PCT/DK02/00894

(51) Int. Cl. A61K 38/36

(52) U.S. Cl. 514/12

(57) ABSTRACT

The invention provides a liquid, aqueous composition, comprising (i) a modified factor VII polypeptide; (ii) an agent suitable for keeping pH in the range of from about 4.0 to about 8.0; (iii) an antioxidant; and (iv) an agent selected from the list of: a calcium salt, a magnesium salt, or a mixture thereof.

### CROSS-REFERENCE TO RELATED APPLICATIONS

This application is a Continuation of U.S. application Ser. No. 10/602,340 filed Jun. 23, 2003 which was a Continuation-in-part of International Application no. PCT/DK02/00894 filed Dec. 20, 2002 and claims priority under 35 U.S.C. 119 of Danish application no. PA 2001 01948 filed Dec. 21, 2001 and Danish application no. PA 2001 01949 filed Dec. 21, 2001 and U.S. application No. 60/346,888 filed Jan. 7, 2002 and U.S. application No. 60/346,399 filed Jan. 7, 2002, and claims priority under 35 U.S.C. 120 of international application no. PCT/DK02/00894 filed Dec. 20, 2002, the contents of which are fully incorporated herein by reference.

### FIELD OF THE INVENTION

The present invention is directed to liquid, aqueous compositions containing modified factor VII polypeptides and to methods for making and using such compositions. More particularly, this invention relates to liquid compositions stabilised against chemical and/or physical degradation.

### BACKGROUND OF THE INVENTION

A variety of factors involved in the blood clotting process have been identified, including factor VII, a plasma glycoprotein. Haemostasis is initiated by the formation of a complex between tissue factor (TF) being exposed to the circulating blood following an injury to the vessel wall, and FVIIa which is present in the circulation in an amount corresponding to about 1% of the total FVII protein mass. FVII exists in plasma mainly as a single-chain zymogen, which is cleaved by FXa into its two-chain, activated form, FVIIa. Recombinant activated factor VIIa (rFVIIa) has been developed as a pro-haemostatic agent.

Modified factor VII molecules are derivatives of the blood coagulation factor VII wherein the molecule (e.g., the catalytic site) has been modified such that the catalytic activity of the active form, factor VIIa, is decreased, while the ability of binding to tissue factor is maintained. Such modified factor VII molecules have been described in WO 92/15686, WO 94/27631, WO 96/12800 and WO 97/47651. Thus, in similarity to the native factor VII molecule, the modified factor VII will bind to tissue factor, but conversely to native factor VIIa, the modified factor VII will not activate the subsequent steps in the extrinsic pathway of coagulation. Thereby, the modified factor VII merely acts as an inhibitor of the formation of a fibrin clot. Therefore, modified factor VIIa molecules have been suggested in the treatment of vascular injury by blocking the production of thrombin and the subsequent deposition of fibrin (WO 97/47651).

As a protein, the modified factor VII molecules are susceptible to physical degradation, including denaturation and aggregation such as the formation of soluble or insoluble aggregates in the form of dimers, oligomers and polymers, or to chemical degradation, including for example, hydrolysis, deamidation and oxidation. The overall consequence is loss of activity of the modified factor VII molecule, formation of toxic and immunogenic degradation products, serious risk of introducing thrombosis upon injection of the degraded modified factor VII molecule, clogging of needles used for injections and risk of non-homogeneity. Thus safety and efficacy of medicaments comprising modified factor VII is directly related to the stability of modified factor VII.

Thus, compositions comprising modified factor VII molecules need to be stabilised. In particularly there is a need for storing and handling medicaments comprising modified factor VII without the requirement of a freezer and wherein the compositions can be stored for a prolonged time such as for at least 6 months before use.

It is desirable to have finished administration forms of modified factor VIIa, suitable for both storage and for delivery. Ideally, the drug product is stored and administered as a liquid. Alternatively, the drug product is lyophilized, i.e., freeze-dried, and then reconstituted by adding a suitable diluent just prior to patient use. Ideally, the drug product has sufficient stability to be kept in long-term storage, i.e., more than six months.

The decision to either maintain the finished drug product as a liquid or to freeze-dry it is usually based on the stability of the protein drug in those forms. Protein stability can be affected inter alia by such factors as ionic strength, pH, temperature, repeated cycles of freeze/thaw, and exposures to shear forces. Active protein may be lost as a result of physical instabilities, including denaturation and aggregation (both soluble and insoluble aggregate formation), as well as chemical instabilities, including, for example, hydrolysis, deamidation, and oxidation, to name just a few. For a general review of stability of protein pharmaceuticals, see, for example, Manning, et al., Pharmaceutical Research 6:903-918 (1989).

While the possible occurrence of protein instabilities is widely appreciated, it is impossible to predict particular instability problems of a particular protein. Any of these instabilities can result in the formation of a protein by-product, or derivative, having lowered activity, increased toxicity, and/or increased immunogenicity. Indeed, protein precipitation may lead to thrombosis, non-homogeneity of dosage form and amount, as well as clogged syringes. Furthermore, post-tranlational modifications such as, for example, gamma carboxylation of certain glutamic acid residues in the N-terminus and addition of carbohydrate side chains provide potential sites that may be susceptible to modification upon storage. Thus, the safety and efficacy of any pharmaceutical composition of a protein is directly related to its stability. Maintaining stability in a liquid dosage form is generally different from a lyophilized dosage form because of greatly increased potential for molecular motion and therefore increased probability of molecular interactions. Maintaining stability in a concentrated form is also different because of the propensity for aggregate formation at increased protein concentrations.

When developing a liquid composition, many factors are taken into consideration. Short-term, i.e., less than six months, liquid stability generally depends on avoiding gross structural changes, such as denaturation and aggregation. These processes are described in the literature for a number of proteins, and many examples of stabilizing agents exist. It is well known that an agent effective at stabilizing one protein actually acts to destabilize another. Once the protein has been stabilized against gross structural changes, developing a liquid composition for long-term stability (e.g., greater than six months) depends on further stabilizing the protein from types of degradation specific to that protein. More specific types of degradation may include, for example, disulfide bond scrambling, oxidation of certain residues, deamidation, cyclization. Although it is not always possible to pinpoint the individual degradation species, assays are developed to monitor subtle changes so as to monitor the ability of specific excipients to uniquely stabilize the protein of interest.

In addition to stability considerations, one generally selects excipients, which are approved by various worldwide medical regulatory agencies. It may be desirable that the composition is approximately isotonic and that the pH of the composition is in a physiologically suitable range upon injection/infusion, otherwise pain and discomfort for the patient may result.

For a general review of protein compositions, see, for example, Cleland et al.: The development of stable protein compositions: A closer look at protein aggregation, deamidation and oxidation, Critical Reviews in Therapeutic Drug Carrier Systems 1993, 10(4): 307-377; and Wang et al., Parenteral compositions of proteins and peptides: Stability and stabilizers, Journal of Parenteral Science and Technology 1988 (Supplement), 42 (2S).

Other publications of interest regarding stabilization of proteins are as follows.
U.S. 20010031721 A1 (American Home Products) concerns highly concentrated, lyophilised, and liquid factor IX compositions.
U.S. Pat. No. 5,770,700 (Genetics Institute) concerns liquid factor IX compositions.
WO 97/19687 (American Red Cross) concerns liquid compositions of plasma proteins, in particular factor VIII and factor IX.
U.S. Pat. No. 4,297,344 discloses stabilization of coagulation factors II and VIII, antithrombin III, and plasminogen against heat by adding selected amino acids such as glycine, alanine, hydroxyproline, glutamine, and aminobutyric acid, and a carbohydrate such as a monosaccharide, an oligosaccharide, or a sugar alcohol.

The development of an aqueous composition for modified factor VII has the advantages of eliminating reconstitution errors, thereby increasing dosing accuracy, as well as simplifying the use of the product clinically, thereby increasing patient compliance. Ideally, compositions of modified factor VIIa should be stable for more than 6 months over a wide range of protein concentrations. This allows for flexibility in methods of administration. Generally, concentrated forms allow for the administration of lower volumes, which is highly desirable from the patients' point of view. Liquid compositions can have many advantages over freeze-dried products with regard to ease of administration and use.

Modified factor VII can today be provided in a liquid formulation, which needs to be stored frozen at −80° C.

Accordingly, there is a need in the art for methods for improving stability of modified factor VII polypeptides and for providing liquid compositions suitable for prolonged storage for more than 6 months at 2 to 8° C. Thus, it is an objective of this invention to provide an aqueous modified factor VII polypeptide composition which provides acceptable control of degradation products.

### SUMMARY OF THE INVENTION

We have discovered that modified factor VII or analogues thereof (“modified factor VII polypeptides”), when formulated in aqueous solution together with an agent suitable for keeping pH in the range of from about 4.0 to about 8.0, an antioxidant and a calcium salt are physically and chemically stable.

In one aspect, the invention provides a liquid, aqueous composition, comprising (i) a modified factor VII polypeptide; (ii) an agent suitable for keeping pH in the range of from about 4.0 to about 8.0; (iii) an antioxidant; and (iv) an agent selected from the list of: a calcium salt, a magnesium salt, or a mixture thereof.

In different embodiments thereof, the pH is kept in the range of from about 4.0 to about 7.0, such as from about 4.5 to about 7.0, from about 5.0 to about 7.0, from about 5.5 to about 7.0, or from about 6.0 to about 7.0.

In one embodiment, the antioxidant (iii) is selected from the list of: L- or D-methionine, a methionine analogue, a methionine-containing peptide, a methionine-homologue, ascorbic acid, cysteine, homocysteine, gluthatione, cystine, and cysstathionine; preferably, the antioxidant is L-methionine.

In different embodiments, the antioxidant is present in a concentration of from about 0.1 to about 5.0 mg/ml, such as from about 0.1 to about 4 mg/ml, from about 0.1 to about 3 mg/ml, from about 0.1 to about 2 mg/ml, or from about 0.5 to about 2 mg/ml.

In a further embodiment, aspect, the composition further comprises (v) a tonicity modifying agent. In one embodiment thereof, the tonicity modifying agent (v) is selected from the list of: a neutral salt; a mono-, di- or polysaccharide; a sugar alcohol; an amino acid; or a small peptide, or a mixture of at least two of said modifying agents. In one, preferred embodiment, the tonicity modifier is mannitol or a neutral salt, preferably sodium chloride. In one embodiment, the tonicity modifying agent (v) is present in a concentration of from 1 mM to 500 mM, such as 10-250 mM.

In another embodiment, the composition further comprises (vi) a non-ionic surfactant. In one embodiment thereof, the non-ionic surfactant (vi) is present in an amount of from 0.005 to 2% by weight. In one embodiment, the non-ionic surfactant is a polysorbate or a poloxamer or a polyoxyethylene alkyl ether such as poloxamer 188, poloxamer 407, polysorbate 20, polysorbate 80, or polyoxy 23 lauryl ether.

In one embodiment of the invention, the agent (ii) suitable for keeping pH in the range of from about 4.0 to about 8.0 is selected from the list of acids and salts of: citrate, acetate, histidine, malate, phosphate, tartaric acid, succinic acid, MES, HEPES, Imidazol, TRIS, lactate, glycylglycin, PIPES, glycin, or a mixture of at least two of said agents. In one embodiment, the concentration of the agent (ii) is from about 1 mM to about 50 mM such as about 10 mM.

In yet one embodiment, the calcium and/or magnesium salt (agent (iv)) is present in a concentration of from about 5 mM to about 150 mM, such as from about 5 mM to about 100 mM, from about 5 mM to about 50 mM, such as from about 10 mM to about 50 mM.

In preferred embodiments, the calcium salt is selected from the list of: calcium chloride, calcium acetate, calcium gluconate, and calcium laevulate, and the magnesium salt is selected from the list of: magnesium chloride, magnesium acetate, magnesium sulphate, magnesium gluconate, and magnesium laevulate.

In a further embodiment, the composition further comprises (vii) a preservative, such as phenol, benzyl alcohol, orto-cresol, meta-cresol, para-cresol, methyl paraben, propyl paraben, benzalconium chloride, or benzaethonium chloride.

In one embodiment, the composition is isotonic. In one embodiment, the composition is formulated for pharmaceutical administration. In one embodiment, the composition is stable and/or stabilized for at least 6 months at 2-8° C.

In one embodiment of the invention, the modified factor VII polypeptide has a biological activity relative to wild-type factor VIIa of less than about 25%, preferably less than about 10%, more preferably less than about 5% and most preferably less than about 1% of the specific activity of wild-type factor VII when tested in one or more of a clotting assay, proteolysis assay, or TF binding assay as described in the present specification.

In one series of embodiments, the modified factor VII polypeptide is selected from the list of: human and bovine factor VII, wherein the active site residue Ser344 is modified, replaced with Gly, Met, Thr, or more preferably, Ala; human factor VII, wherein the residue Lys341 is replaced; human factor VII, wherein the residue Asp242 is replaced; human factor VII, wherein the residue His193 is replaced; FVII-(K341A); FVII-(S344A); FVII-(D242A); FVII-(H193A); a factor VII polypeptide modified in the active site by reaction with a reagent selected from the list of: peptide chloromethylketones or peptidyl cloromethanes; azapeptides; acylating agents such as various guanidinobenzoate derivatives and 3-alkoxy-4- chloroisocoumarins; sulphonyl fluorides such as phenylmethylsulphonylfluoride (PMSF); diisopropylfluorophosphate (DFP); tosylpropylchloromethyl ketone (TPCK); tosylysylchloromethyl ketone (TLCK); nitrophenylsulphonates; heterocyclic protease inhibitors such as isocoumarines, and coumarins; a factor VII polypeptide modified in the active site by reaction with a reagent selected from the list of: L-Phe-Phe-Arg chloromethyl ketone, D-Phe-Phe-Arg chloromethyl ketone, L-Phe-Pro-Arg chloromethyl ketone, D-Phe-Pro-Arg chloromethyl ketone, L-Glu-Gly-Arg chloromethyl ketone, D-Glu-Gly-Arg chloromethyl ketone, Dansyl-L-Phe-Phe-Arg chloromethyl ketone, Dansyl-D-Phe-Phe-Arg chloromethyl ketone, Dansyl-L-Phe-Pro-Arg chloromethyl ketone, Dansyl-D-Phe-Pro-Arg chloromethyl ketone, Dansyl-L-Glu-Gly-Arg chloromethylketone, and Dansyl-D-Glu-Gly-Arg chloromethylketone.

In preferred embodiments, the modified factor VII polypeptide is selected from the list of: FVII-(S344A); FVII-(H193A); and a factor VII polypeptide modified in the active site by reaction with a reagent selected from the list of: L-Phe-Phe-Arg chloromethyl ketone, D-Phe-Phe-Arg chloromethyl ketone, L-Phe-Pro-Arg chloromethyl ketone, D-Phe-Pro-Arg chloromethyl ketone, L-Glu-Gly-Arg chloromethyl ketone, D-Glu-Gly-Arg chloromethyl ketone, Dansyl-L-Phe-Phe-Arg chloromethyl ketone, Dansyl-D-Phe-Phe-Arg chloromethyl ketone, Dansyl-L-Phe-Pro-Arg chloromethyl ketone, Dansyl-D-Phe-Pro-Arg chloromethyl ketone, Dansyl-L-Glu-Gly-Arg chloromethylketone, and Dansyl-D-Glu-Gly-Arg chloromethylketone.chloromethylketone, Dansyl-D-Phe-Pro-Arg chloromethylketone, Dansyl-L-Glu-Gly-Arg chloromethylketone, and Dansyl-D-Glu-Gly-Arg chloromethylketone.

In one series of embodiments, the modified factor VII polypeptide is present in a concentration of from about 0.1 mg/ml to about 15 mg/ml, such as from about 0.5 to about 10 mg/ml, from about 0.5 to about 5.0 mg/ml, or from about 1.0 mg/ml to 5.0 mg/ml.

In another aspect, the invention provides a method for preparing a liquid aqueous composition of a modified factor VII polypeptide, comprising providing a modified factor VII polypeptide in a solution comprising (ii) an agent suitable for keeping pH in the range of from about 4.0 to about 8.0; (iii) an antioxidant; and (iv) an agent selected from the list of: a calcium salt, a magnesium salt, or a mixture thereof.

In another aspect, the invention concerns the use of the composition for the preparation of a medicament for inhibiting blood clotting. In another aspect, the invention concerns the use of the composition for the preparation of a medicament for inhibiting tissue factor mediated reactions.

In another aspect, the invention concerns a method for inhibiting blood clotting in a subject, the method comprising administering to a subject in need thereof an effective amount of an aqueous liquid composition comprising (i) a modified factor VII polypeptide, (ii) an agent suitable for keeping pH in the range of from about 4.0 to about 8.0; (iii) an antioxidant; and (iv) an agent selected from the list of: a calcium salt, a magnesium salt, or a mixture thereof.

In yet another aspect, the invention concerns a method for inhibiting tissue factor mediated reactions in a subject, the method comprising administering to a subject in need thereof an effective amount of an aqueous liquid composition comprising (i) a modified factor VII polypeptide, (ii) an agent suitable for keeping pH in the range of from about 4.0 to about 8.0; (iii) an antioxidant; and (iv) an agent selected from the list of: a calcium salt, a magnesium salt, or a mixture thereof.

In different embodiments, the unwanted blood clotting is associated with a condition selected from the group consisting of: angioplasty, deep vein thrombosis, pulmonary embolism, stroke, disseminated intravascular coagulation (DIC), fibrin deposition in tissue, e.g., in lungs and/or kidneys associated with gram-negative endotoxemia, and myocardial infarction.

In different embodiments, the tissue factor mediated reactions are associated with a condition selected from the group consisting of Systemic Inflammatory Response Syndrome (SIRS), Acute Respiratory Disease Syndrome (ARDS), Multiple Organ Failure (MOF), HUS, and TTP.

### DETAILED DESCRIPTION OF THE INVENTION

The compositions according to the present invention are useful as stable and preferably ready-to-use compositions of modified factor VII polypeptides. The compositions are stable for at least six months, and preferably up to 36 months; when stored at temperatures ranging from 2° to 8° C. The compositions are chemically and/or physically stable, in particular chemically stable, when stored for at least 6 months at from 2° to 8° C.

“Stable” is intended to mean that the composition, after storage for 6 months at 2 to 8° C. retains at least 50% of its initial biological activity as measured by a competition clot assay essentially as described in WO 92/15686 (Example III) or in one or more of the Assays 5 to 8 as described in the present specification (see “assay” part, below). Preferably, the stable composition retains at least 80% of its initial activity after storage for 6 months at 2 to 8° C.

The term “physically stable” is intended to designate a composition which remains visually clear. Physical stability of the compositions is evaluated by means of visual inspection and turbidity after storage of the composition at different temperatures for various time periods. Visual inspection of the compositions is performed in a sharp focused light with a dark background. A composition is classified physical unstable, when it shows visual turbidity.

The term “physical stability” of modified factor VII polypeptides relates to the formation of insoluble and/or soluble aggregates in the form of dimeric, oligomeric and polymeric forms of modified factor VII polypeptides as well as any structural deformation and denaturation of the molecule.

The term “chemically stable” is intended to designate a composition which retains at least 50% of its initial biological activity after storage for 6 months at 2 to 8° C., as measured by a competing clot assay essentially as described in WO 92/15686 (Example III) or in one or more of the Assays 5 to 8 as described in the present specification (see “assay” part, below).

The term “chemical stability” is intended to relate to the formation of any chemical change in the modified factor VII polypeptides upon storage in dissolved or solid state at accelerated conditions. By example are hydrolysis, deamidation and oxidation. In particular, the sulphur-containing amino acids are prone to oxidation with the formation of the corresponding sulphoxides.

The compositions comprise modified factor VII polypeptides, antioxidants, calcium and/or magnesium ions, buffering agents, and, optionally, other excipients, which further stabilize the modified factor VII polypeptides, including tonicity modifiers. The modified factor VII polypeptides concentration ranges from about 0.1 to about 15 mg/mL.

As used herein, the term “tonicity modifier” includes agents, which contribute to the osmolality of the solution. Tonicity modifiers include, but are not limited to, amino acids; small peptides (e.g., having from 2 to 5 amino acid residues); neutral salts; mono- or disaccharides; polysaccharides; sugar alcohols, or a mixture of at least two of said modifiers. Examples of tonicity modifiers include, but are not limited to, sodium chloride, potassium chloride, sodium citrate, sucrose, glucose, glycylglycine, and mannitol. Normally, the modifiers are present at a concentration of from about 1 to about 500 mM; from about 1 to about 300 mM; from about 10 to about 200 mM; or from about 20 to about 150 mM, depending on the other ingredients present. Neutral salts such as, e.g., sodium chloride or potassium chloride may be used. By “neutral salt” is meant a salt that is neither an acid nor a base when dissolved in aqueous solution.

The term “agent suitable for keeping the pH in the range of about 4.0 to about 8.0” encompasses those agents, which maintain the solution pH in an acceptable range-from about 4.0 to about 8.0, such as from about 4.0 to about 7.0, from about 4.5 to about 7.0, from about 5.0 to about 7.0, from about 5.0 to about 6.5, from about 5.5 to about 7.0, from about 5.5 to about 6.5, from about 6.0 to about 7.0, from about 5.0 to about 6.0, from about 6.4 to about 6.6, or about 6.5, from about 5.2 to about 5.7, or about 5.5. The term may be used interchangeably with “buffering agent” These may include, but are not limited to, acids and salt of: citrate (sodium or potassium), acetate (ammonium, sodium or calcium), histidine (L-histidine), malate, phosphate (sodium or potassium), tartaric acid, succinic acid, MES, HEPES, imidazol, TRIS, lactate, glutamate, glycylglycin, PIPES, glycin, or a mixture of at least two of said buffering agents. The buffer concentration range is chosen to maintain the preferred pH of the solution. The buffering agent may also be a mixture of at least two buffering agents, wherein the mixture is able to provide a pH value in the specified range. In alternative embodiments, the buffer concentration is in the range of from about 1 mM to 100 mM; from 1 mM to about 50 mM; from about 1 mM to about 25 mM; from about 2 mM to about 20 mM; or about 10 mM.

Optionally, the compositions may also contain a surfactant or detergent. “Surfactants” or “detergents” generally include those agents which protect the protein from air/solution interface induced stresses and solution/surface induced stresses (e.g., resulting in protein aggregation). The detergent is preferably a non-ionic detergent including, but not limited to polysorbates (e.g. Tween®), such as polysorbate 20 or 80; polyoxyethylene alkyl ethers or poloxamers, such as poloxamer 188 or 407, (e.g., Pluronic® polyols) and other ethylene/polypropylene block polymers, or polyethyleneglycol (PEG) such as PEG8000. The amount of surfactant present ranges from about 0.005 to 2%.

The composition also includes an antioxidant. Antioxidants include, but are not limited to, ascorbic acid, cysteine, homocysteine, cystine, cysstathionine, methionine, gluthatione, and other peptides containing cysteine or methionine in particular peptides with 2 to 5 amino acid residues wherein at least one of the residues is a methionine or cysteine residue; methionine, in particular L-methionine, is preferred. The antioxidant is included at a concentration of 0.1 to 5 mg/ml, such as 0.1 to 4, 0.1 to 3, 0.1 to 2, or 0.5 to 2 mg/ml.

A preservative may also be included in the composition to retard microbial growth and thereby allow “multiple use” packaging of the FVII polypeptides. Preservatives include phenol, benzyl alcohol, orto-cresol, meta-cresol, para-cresol, methyl paraben, propyl paraben, benzalconium chloride, and benzethonium chloride. The preservative is normally included at a concentration of 0.1 to 20 mg/ml depending on the pH range and the type of preservative. Optionally, the composition may also include an agent capable of inhibiting deamidation.

As used herein, amounts specified are understood to be ± about 10%, e.g., about 50 mM includes 50 mM±5 mM; e.g., 4% includes 4%±0.4%, etc.

Percentages are (weight/weight) both when referring to solids dissolved in solution and liquids mixed into solutions. For example, for Tween, it is the weight of 100% stock/weight of solution.

The term “isotonic” means “isotonic with serum”, i.e., at about 300±50 milliosmol/kg. The tonicity is meant to be a measure of osmolality of the solution prior to administration.

The term “pharmaceutically effective amount” or “effective amount” is the effective dose to be determined by a qualified practitioner, who may titrate dosages to achieve the desired response. Factors for consideration of dose will include potency, bioavailability, desired pharmacokinetic/pharmacodynamic profiles, condition of treatment, patient-related factors (e.g. weight, health, age, etc.), presence of co-administered medications (e.g., other anticoagulants), time of administration, or other factors known to a medical practitioner.

The term “treatment” is defined as the management and care of a subject, e.g. a mammal, in particular a human, for the purpose of combating the disease, condition, or disorder and includes the administration of a modified factor VII polypeptide to prevent the onset of the symptoms or complications, or alleviating the symptoms or complications, or eliminating the disease, condition, or disorder. Pharmaceutical compositions according to the present invention containing a modified factor VII polypeptide may be administered parenterally to subjects in need of such a treatment. Parenteral administration may be performed by subcutaneous, intramuscular or intravenous injection by means of a syringe, optionally a pen-like syringe. Alternatively, parenteral administration can be performed by means of an infusion pump.

Methods of Use:

Preparations according to the invention, comprising modified factor VII polypeptides, which have substantially reduced bioactivity relative to wild-type factor VII, may be used as anticoagulants, such as, e.g., in patients undergoing angioplasty or other surgical procedures that may increase the risk of thrombosis or occlusion of blood vessels as occurs, e.g., in restenosis. Other medical indications for which anticoagulants are prescribed include, without limitation, deep vein thrombosis, pulmonary embolism, stroke, disseminated intravascular coagulation (DIC), fibrin deposition in tissues such as e.g., in lungs and/or kidneys associated with gram-negative endotoxemia, myocardial infarction; Acute Respiratory Distress Syndrome (ARDS), Systemic Inflammatory Response Syndrome (SIRS), Hemolytic Uremic Syndrome (HUS), MOF, and TTP.

Factor VII Polypeptides to be Formulated According to the Present Invention:

The terms “human factor VII” or “FVII” denote human factor VII produced by methods including natural source extraction and purification, and by recombinant cell culture systems. Its sequence and characteristics are set forth, for example, in U.S. Pat. No. 4,784,950. The term “factor VII” is intended to encompass factor VII polypeptides in their uncleaved (zymogen) form, as well as those that have been proteolytically processed to yield their respective bioactive forms, which may be designated factor VIIa. Typically, factor VII is cleaved between residues 152 and 153 to yield factor VIIa. The term “factor VII” is also intended to encompass, without limitation, polypeptides having the amino acid sequence 1-406 of wild-type human factor VII (as disclosed in U.S. Pat. No. 4,784,950), as well as wild-type factor VII derived from other species, such as, e.g., bovine, porcine, canine, murine, and salmon factor VII. It further encompasses natural allelic variations of factor VII that may exist and occur from one individual to another. Also, degree and location of glycosylation or other post-translation modifications may vary depending on the chosen host cells and the nature of the host cellular environment.

As used herein, “modified factor VII polypeptides” encompasses, without limitation, polypeptides in which the factor VII biological activity has been substantially modified or reduced relative to the activity of wild-type human factor VIIa. These polypeptides include, without limitation, factor VII or factor VII that has been chemically modified and factor VII variants into which one or more specific amino acid sequence alterations have been introduced that modify or disrupt the bioactivity of the polypeptide. The term is intended to cover substitution, deletion and insertion amino acid variants of factor VII or posttranslational modifications. Modified factor VII exhibiting substantially modified or reduced bioactivity relative to wild-type factor VII, encompasses, without limitation, factor VII polypeptides that have either been chemically modified relative to human factor VII and/or contain one or more amino acid sequence alterations relative to human factor VII (i.e., factor VII variants), and/or contain truncated amino acid sequences relative to human factor VII (i.e., factor VII fragments).

Modified factor VII further emcompasses polypeptides having a modified N-terminal end including N-terminal amino acid deletions or additions.

Modified factor VII polypeptides, including variants, having substantially reduced biological activity relative to wild-type factor VII are those that exhibit less than about 25%, preferably less than about 10%, more preferably less than about 5% and most preferably less than about 1% of the specific activity of wild-type factor VII that has been produced in the same cell type when tested in one or more of a clotting assay, proteolysis assay, or TF binding assay as described in Assays 1 to 4 (see “assay” part, below).

The term “modified factor VII polypeptides”, as used herein, is intended to mean factor VII polypeptides having at least one modification, which modification substantially inhibits the ability of the modified factor VII to activate plasma factor X or factor IX. This includes, without limitation, factor VII polypeptides having substantially reduced catalytic activity, as well as fragments thereof. The inactive factor VII polypeptides bind to tissue factor with high affinity and specificity but do not initiate blood coagulation. The terms “catalytically inactive factor VII polypeptides”, “inactive factor VII polypeptides”, or “FVIIai” may be used interchangeably with “modified factor VII polypeptides” or “modified factor VII”.

In one embodiment of the invention, modified factor VII polypeptides encompass those that exhibit at least about 10%, at least about 20%, at least about 25%, at least about 30%, at least about 40%, at least about 50%, at least about 60%, at least about 70%, at least about 75%, at least about 80%, at least about 90%, at least about 100%, at least about 110%, at least about 120%, or at least about 130%, of the specific TF-binding affinity of wild-type factor VIIa, when tested in one or more of the TF binding assays as described in the present specification. In a preferred embodiment, the TF antagonists exhibit at least about 75% of the binding affinity of wild-type factor VIIa. The term “TF binding activity” as used herein means the ability of a FVIIa polypeptide or TF antagonist to inhibit the binding of recombinant human 125I-FVIIa to cell surface human TF. The TF binding activity may be measured as described in Assay 3 (of the present specification).

In another embodiment, modified factor VII polypeptides encompass those that exhibit less than about 25%, more preferably less than about 10%, or 5%, or 3%, or 2%, and most preferably less than about 1% of the specific activity of wild-type factor VII, when tested in one or more of a clotting assay, or proteolysis assay as described in Assays 1 to 4 of the present specification.

Non-limiting examples of factor VII variants having substantially reduced or modified biological activity relative to wild-type factor VII include R152E-FVIIa (Wildgoose et al., Biochem 29:3413-3420, 1990), S344A-FVIIa (Kazama et al., J. Biol. Chem. 270:66-72, 1995), FFR-FVIIa (Holst et al., Eur. J. Vasc. Endovasc. Surg. 15:515-520, 1998), and factor VII lacking the Gla domain, (Nicolaisen et al., FEBS Letts. 317:245-249, 1993). Non-limiting examples also include human FVIIa, which has the lysine residue in position 341 replaced by another amino acid residue; human FVIIa, which has the serine residue in position 344 replaced by another amino acid residue; human FVIIa, which has the aspartic acid residue in position 242 replaced by another amino acid residue; human FVIIa, which has the histidine residue in position 193 replaced by another amino acid residue; FVII-(K341A); FVII-(S344A); FVII-(D242A); and FVII-(H193A). Non-limiting examples of chemically modified factor VII polypeptides and sequence variants are described, e.g., in U.S. Pat. No. 5,997,864.

The catalytic activity of factor VIIa may be inhibited by chemical derivatization of the catalytic center, or triad. Derivatization may be accomplished by reacting factor VII with an irreversible inhibitor such as an organophosphor compound, a sulfonyl fluoride, a peptide halomethyl ketone or an azapeptide, or by acylation, for example, peptide chloromethylketones or peptidyl cloromethanes; azapeptides; acylating agents such as various guanidinobenzoate derivatives and 3-alkoxy-4-chloroisocoumarins; sulphonyl fluorides such as phenylmethylsulphonylfluoride (PMSF); diisopropylfluorophosphate (DFP); tosylpropylchloromethyl ketone (TPCK); tosylysylchloromethyl ketone (TLCK); nitrophenylsulphonates; heterocyclic protease inhibitors such as isocoumarines, and coumarins.

Preferred peptide halomethyl ketones include Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone and Dansyl-D-Glu-Gly-Arg chloromethylketone.

In preferred embodiments, amino acid substitutions are made in the amino acid sequence of the factor VII catalytic triad, defined herein as the regions which contain the amino acids which contribute to the factor VIIa catalytic site. The substitutions, insertions or deletions in the catalytic triad are generally at or adjacent to the amino acids which form the catalytic site. In the human and bovine factor VII proteins, the amino acids which form a catalytic “triad” are Ser344, Asp242, and His193 (subscript numbering indicating position in human wild type factor VII). The catalytic sites in factor VII from other mammalian species may be determined using presently available techniques including, among others, protein isolation and amino acid sequence analysis. Catalytic sites may also be determined by aligning a sequence with the sequence of other serine proteases, particularly chymotrypsin, whose active site has been previously determined (Sigler et al., J. Mol. Biol., 35:143-164 (1968), incorporated herein by reference), and therefrom determining from said alignment the analogous active site residues.

The amino acid substitutions, insertions or deletions are made so as to prevent or otherwise inhibit activation by the factor VII of factors X and/or IX. The factor VII so modified should, however, also retain the ability to compete with authentic factor VII and/or factor VIIa for binding to tissue factor in the coagulation cascade. Such competition may readily be determined by means of, e.g., a clotting assay as described herein, or a competition binding assay using, e.g., a cell line having cell-surface tissue factor, such as the human bladder carcinoma cell line J82 (Sakai et al. J. Biol. Chem. 264: 9980-9988 (1989)).

The amino acids which form the catalytic site in factor VII, such as Ser344, Asp242, and His193 in human and bovine factor VII, may either be substituted or deleted. It is preferred to change only a single amino acid, thus minimizing the likelihood of increasing the antigenicity of the molecule or inhibiting its ability to bind tissue factor, however two or more amino acid changes (substitutions, additions or deletions) may be made and combinations of substitution(s), addition(s) and deletion(s) may also be made. In a preferred embodiment for human and bovine factor VII, Ser344 is preferably substituted with Ala, but Gly, Met, Thr or other amino acids can be substituted. It is preferred to replace Asp with Glu and to replace His with Lys or Arg. In general, substitutions are chosen to disrupt the tertiary protein structure as little as possible. One may introduce residue alterations as described above in the catalytic site of appropriate factor VII sequence of human, bovine or other species and test the resulting protein for a desired level of inhibition of catalytic activity and resulting anticoagulant activity as described herein.

In preferred embodiments of human and bovine factor VII, the active site residue Ser344 is modified, replaced with Gly, Met, Thr, or more preferably, Ala. Such substitution could be made separately or in combination with substitution(s) at other sites in the catalytic triad, which includes His193 and Asp242.

Biological Activity of Factor VII Polypeptides:

The biological activity of factor VIIa in blood clotting derives from its ability to (i) bind to tissue factor (TF) and (ii) catalyze the proteolytic cleavage of factor IX or factor X to produce activated factor IX or X (factor IXa or Xa, respectively).

For purposes of the invention, biological activity of factor VII polypeptides (“factor VII biological activity”) may be quantified by measuring the ability of a preparation to promote blood clotting using factor VII-deficient plasma and thromboplastin, as described, e.g., in U.S. Pat. No. 5,997,864 or WO 92/15686. In this assay, biological activity is expressed as the reduction in clotting time relative to a control sample and is converted to “factor VII units” by comparison with a pooled human serum standard containing 1 unit/ml factor VII activity. Alternatively, factor VII biological activity may be quantified by
Measuring the ability of factor VIIa or a factor VIIa equivalent to produce activated factor X (factor Xa) in a system comprising TF embedded in a lipid membrane and factor X. (Persson et al., J. Biol. Chem. 272:19919-19924, 1997);
Measuring factor X hydrolysis in an aqueous system (“In Vitro Proteolysis Assay”, see below);
Measuring the physical binding of factor VIIa or a factor VIIa equivalent to TF using an instrument based on surface plasmon resonance (Persson, FEBS Letts. 413:359-363, 1997); and
Measuring hydrolysis of a synthetic substrate by factor VIIa and/or a factor VIIa equivalent (“In Vitro Hydrolysis Assay”, see below); and
Measuring generation of thrombin in a TF-independent in vitro system.
   Assays Suitable for Determining Biological Activity of Factor VII Polypeptides:

Factor VII polypeptides useful in accordance with the present invention may be selected by suitable assays that can be performed as simple preliminary in vitro tests. Thus, the present specification discloses a simple test (entitled “In Vitro Hydrolysis Assay”) for the activity of factor VII polypeptides.

In Vitro Hydrolysis Assay (Assay 1)

Native (wild-type) factor VIIa and factor VII polypeptide (both hereafter referred to as “factor VIIa”) may be assayed for specific activities. They may also be assayed in parallel to directly compare their specific activities. The assay is carried out in a microtiter plate (MaxiSorp, Nunc, Denmark). The chromogenic substrate D-IIe-Pro-Arg-p-nitroanilide (S-2288, Chromogenix, Sweden), final concentration 1 mM, is added to factor VII (final concentration 100 nM) in 50 mM Hepes, pH 7.4, containing 0.1 M NaCl, 5 mM CaCl2 and 1 mg/ml bovine serum albumin. The absorbance at 405 nm is measured continuously in a SpectraMax™ 340 plate reader (Molecular Devices, USA). The absorbance developed during a 20-minute incubation, after subtraction of the absorbance in a blank well containing no enzyme, is used to calculate the ratio between the activities of factor VII polypeptide and wild-type factor VIIa:
Ratio=(A405 nm factor VII polypeptide)/(A405 nm factor VIIa wild-type).

Based thereon, factor VII polypeptides with an activity lower than, comparable to, or higher than native factor VIIa may be identified, such as, for example, factor VII polypeptides where the ratio between the activity of the factor VII polypeptide and the activity of native factor VII (wild-type FVII) is about, versus above 1.0.

The activity of the factor VII polypeptides may also be measured using a physiological substrate such as factor X (“In Vitro Proteolysis Assay”), suitably at a concentration of 100-1000 nM, where the factor Xa generated is measured after the addition of a suitable chromogenic substrate (eg. S-2765). In addition, the activity assay may be run at physiological temperature.

In Vitro Proteolysis Assay (Assay 2)

Native (wild-type) factor VIIa and factor VII polypeptide (both hereafter referred to as “factor VIIa”) are assayed in parallel to directly compare their specific activities. The assay is carried out in a microtiter plate (MaxiSorp, Nunc, Denmark). Factor VII (10 nM) and factor X (0.8 microM) in 100 microL 50 mM Hepes, pH 7.4, containing 0.1 M NaCl, 5 mM CaCl2 and 1 mg/ml bovine serum albumin, are incubated for 15 min. Factor X cleavage is then stopped by the addition of 50 microL 50 mM Hepes, pH 7.4, containing 0.1 M NaCl, 20 mM EDTA and 1 mg/ml bovine serum albumin. The amount of factor Xa generated is measured by addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax™ 340 plate reader (Molecular Devices, USA). The absorbance developed during 10 minutes, after subtraction of the absorbance in a blank well containing no FVIIa, is used to calculate the ratio between the proteolytic activities of factor VII polypeptide and wild-type factor VIIa:
Ratio=(A405 nm factor VII polypeptide)/(A405 nm factor VII wild-type).

Based thereon, factor VII polypeptide with an activity lower than, comparable to, or higher than native factor VIIa may be identified, such as, for example, factor VII polypeptides where the ratio between the activity of the factor VII polypeptide and the activity of native factor VII (wild-type FVII) is about, versus above 1.0.

The ability of factor VIIa or factor VII polypeptides to generate thrombin can also be measured in an assay (assay 3) comprising all relevant coagulation factors and inhibitors at physiological concentrations (minus factor VIII when mimicking hemophilia A conditions) and activated platelets (as described on p. 543 in Monroe et al. (1997) Brit. J. Haematol. 99, 542-547, which is hereby incorporated as reference).

The activity of the factor VII polypeptides may also be measured using a one-stage clot assay (assay 4) essentially as described in WO 92/15686 or U.S. Pat. No. 5,997,864. Briefly, the sample to be tested is diluted in 50 mM Tris (pH 7.5), 0.1% BSA and 100 μl is incubated with 100 μl of factor VII deficient plasma and 200 μl of thromboplastin C containing 10 mM Ca²⁺. Clotting times are measured and compared to a standard curve using a reference standard or a pool of citrated normal human plasma in serial dilution.

Competition Assays:

Inhibition of FVIIa/sTF Amidolytic Activity (Assay 5):

Inhibition of FVIIa-TF catalyzed amidolytic activity by modified factor VII is tested employing soluble human TF (10 nM), recombinant human FVIIa (10 nM) and increasing concentrations of modified factor VII. Varying concentrations of the modified factor VII are preincubated with 10 nM sTF and 10 nM FVIIa in BSA buffer (50 mM Hepes, pH 7.4, 100 mM NaCl, 5 mM CaCl2 and 1 mg/ml BSA) for 60 min at room temperature before addition of substrate S2288 (1.2 mM, Chromogenix). The colour development is measured continuously for 30 min at 405 nm. Amidolytic activity is presented as mOD/min. IC50 values for inhibition of FVIIa/TF amidolytic activity by the modified factor VII may be calculated.

Inhibition of FXa Generation (Assay 6).

Lipidated TF (10 pM), FVIIa (100 pM) and modified factor VII (0-50 nM) in BSA buffer (see assay 4) are incubated 60 min at room temperature before FX (50 nM ) is added. The reaction is stopped after another 10 min by addition of ½ volume stopping buffer (50 mM Hepes, pH 7.4, 100 mM NaCl, 20 mM EDTA). The amount of FXa generated is determined by adding substrate S2765 (0.6 mM, Chromogenix, and measuring absorbance at 405 nm continuously for 10 min. IC50 values for modified factor VII-inhibition of FVIIa/lipidated TF-mediated activation of FX may be calculated.

TF-Dependent Clotting Assay (Assay 7):

The assay is carried out on an ACL300 Research clotting apparatus (ILS Laboratories). Dilutions of modified factor VII in 50 mM imidazole, pH 7.4, 100 mM NaCl, 0.1% BSA are mixed with 25 mM CaCl2 in the ratio of 2 to 5 and added to sample cups in the clotting apparatus. Thromboplastin from human, rat, rabbit, baboon, or pig diluted with the imidazole buffer to give clotting time of approximately 30 sec in samples without modified factor Vii is placed in reagent reservoir 2, and human, rat, rabbit, baboon, or pig plasma, in reagent reservoir 3. During the analysis 70 μl of the modified factor VII and CaCl2 mixture is transferred to 25 μl thromboplastin reagent and preincubated 900 sec before addition of 60 pi plasma and measuring of the clotting time. Maximal clotting time is set to 400 sec. A dilution of the thromboplastin is used as standard curve for converting clotting times into TF activity relative to the control without modified FVII added.

Inhibition of FVIIa/Cell Surface TF Catalyzed Activation of FX by Modified Factor VII (Assay 8):

Monolayers of cells expressing human TF, e.g. human lung fibroblasts WI-38 (ATTC No. CCL-75), human bladder carcinoma cell line J82 (ATTC No. HTB-1), human keratinocyte cell line CCD 1102KerTr (ATCC no. CRL-2310), human glioblastoma cell line U87, or human breast cancer cell line MDA-MB231, are employed as TF source in FVIIa/TF catalyzed activation of FX. Confluent cell monolayers in a 24-, 48- or 96-well plate are washed one time in buffer A (10 mM Hepes, pH 7.45, 150 mM NaCl, 4 mM KCl, and 11 mM glucose) and one time in buffer B (buffer A supplemented with 1 mg/ml BSA and 5 mM Ca2+). FVIIa (1 nM), FX (135 nM) and varying concentrations of modified factor VII in buffer B are simultaneously added to the cells. Alternatively the cells are preincubated 15 min with modified factor VII before addition of rFVIIa and FX. FXa formation is allowed for 15 min at 37° C. 50 μl aliquots are removed from each well and added to 50 μl stopping buffer (Buffer A supplemented with 10 mM EDTA and 1 mg/ml BSA). The amount of FXa generated is determined by transferring 50 μl of the above mixture to a microtiter plate well and adding 25 μl Chromozym X (final concentration 0.6 mM) to the wells. The absorbance at 405 nm is measured continuously and the initial rates of colour development are converted to FXa concentrations using a FXa standard curve.

Preparation and Purification of Modified Factor VII Polypeptides:

Modified factor VII molecules suitable to be formulated according to the present invention and the manufacture thereof have been described in WO 92/15686, WO 94/27631, WO 96/12800 and WO 97/47651.

In general, human purified factor VIIa is preferably made by DNA recombinant technology, e.g. as described by Hagen et al., Proc. Natl. Acad. Sci. USA 83: 2412-2416, 1986, or as described in European Pat. No. 200.421 (ZymoGenetics, Inc.).

Factor VII may also be produced by the methods described by Broze and Majerus, J. Biol. Chem. 255 (4):. 1242-1247, 1980 and Hedner and Kisiel, J. Clin. Invest. 71: 1836-1841, 1983. These methods yield factor VII without detectable amounts of other blood coagulation factors. An even further purified factor VII preparation may be obtained by including an additional gel filtration as the final purification step. Factor VII is then converted into activated factor VIIa by known means, e.g. by several different plasma proteins, such as factor XIIa, IXa or Xa. Alternatively, as described by Bjoern et al. (Research Disclosure, 269 September 1986, pp. 564-565), factor VII may be activated by passing it through an ion-exchange chromatography column, such as Mono Q® (Pharmacia fine Chemicals) or the like, or by autoactivation in solution.

The factor VII polypeptide, whether isolated or recombinantly made, may then be chemically modified as described in, e.g., WO 92/15686, WO 94/27631, WO 96/12800 and WO 97/47651, or by Sorensen et al. J. Biol. Chem. 272: 11863-11868, 1997 (FFR-rFVIIa: FVIIa blocked in the active site with D-Phe-L-Phe-L-Arg-chloromethyl ketone).

Factor VII variants may be produced by modification of wild-type factor VII or by recombinant technology. Factor VII equivalents with altered amino acid sequence when compared to wild-type factor VII may be produced by modifying the nucleic acid sequence encoding wild-type factor VII either by altering the amino acid codons or by removal of some of the amino acid codons in the nucleic acid encoding the natural factor VII by known means, e.g. by site-specific mutagenesis (see, e.g., Cunningham and Wells, 1989, Science 244:1081-1085).

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, modified factor VII polypeptides may be further purified. Purification may be achieved using any method known in the art, including, without limitation, affinity chromatography, such as, e.g., on an anti-factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J. C. Janson and Lars Ryden, editors, VCH Publishers, New York, 1989. Following purification, the preparation preferably contains less than about 10% by weight, more preferably less than about 5% and most preferably less than about 1%, of non-factor VII polypeptides derived from the host cell.

Factor VII polypeptides may be turned into its two-chain form by proteolytic cleavage, using factor XIIa or other proteases having trypsin-like specificity, such as, e.g., factor IXa, kallikrein, factor Xa, and thrombin. See, e.g., Osterud et al., Biochem. 11:2853(1972); Thomas, U.S. Pat. No. 4,456,591; and Hedner et al., J. Clin. Invest. 71:1836 (1983). Alternatively, factor VII polypeptides may be activated by passing it through an ion-exchange chromatography column, such as Mono Q® (Pharmacia) or the like, or by autoactivation in solution. The resulting polypeptide may then be formulated and administered as described in the present application.

The following examples illustrate practice of the invention. These examples are for illustrative purposes only and are not intended in any way to limit the scope of the invention claimed.

### EXPERIMENTAL EXAMPLES

### Example 1

A. Assay Methods

The content of aggregates is determined by non-denaturing size exclusion HPLC. The content of oxidized forms is determined by RP-HPLC. The content of enzymatic degradation forms is determined by RP-HPLC.

Nondenaturing size exclusion chromatography was run on a Waters Protein Pak 300 SW column, 7.5×300 mm using 0.2 M ammoniumsulfat, 5% 2-propanol pH 7.0 as mobile phase. Flow rate :0.5 ml/min. Detection: 215 nm. Load: 25 μg FVIIa.

Reverse phase HPLC was run on a proprietary 4.5×250 mm butylbonded silica column with a particle size of 5 μm and pore size 300 Å. Column temperature: 70° C. A-buffer: 0.1% v/v trifluoracetic acid. B-buffer: 0.09% v/v trifluoracetic acid, 80% v/v acetonitrile. The column was eluted with a linear gradient from X to (X+13)% B in 30 minutes. X is adjusted so that FVIIa elutes with a retention time of approximately 26 minutes. Flow rate: 1.0 ml/min. Detection: 214 nm. Load: 25 μg FVIIa.

### Example 2

Chemical Stability of Aqueous Phe-Phe-Arg Chloromethyl Ketone-Inactivated Factor VII (FFR-rFVIIa) Formulations Containing Methionine as Antioxidant

Two different formulations were prepared. The compositions of the formulations were:

| | | |
|---|---|---|
| FFR-rFVIIa | 2 | mg/ml |
| NaCl | 2.8-2.9 | mg/ml |
| CaCl2, 2 H2O | 1.4-1.5 | mg/ml |
| Glycylglycine | 1.3 | mg/ml |
| Methionine | 0 or 1 | mg/ml |

| | |
|---|---|
| pH | 7.0 |

The formulations were prepared from a liquid bulk solution of FFR-rFVIIa containing FFR-rFVIIa, NaCl, CaCl2 and glycylglycine. The methionine was dissolved in water. The FFR-rFVIIa bulk and the methionine solutions were mixed, and the pH in the solutions was adjusted to 7.0. The formulations were filtered (0.2 μm) and filled in vials (2.2 ml solution per vial). The vials were stored at 35° C. Samples were withdrawn and analysed for content of oxidized forms (by RP-HPLC) at the time points stated in the table below. The table shows the content of oxidised forms (in %).

| Methionine (mg/ml) | Time zero | 35° C. 2 weeks | 35° C. 4 weeks |
|---|---|---|---|
| 0 (reference) | 2.7 | 3.7 | 3.9 |
| 1 | 2.7 | 3.0 | 2.9 |

The results show that addition of methionine slows down the oxidation rate in the formulation.

### Example 3

### Long-Term Stability of an Aqueous Formulation of FFR-rFVIIa

A formulation of the following composition was prepared:

| | | |
|---|---|---|
| FFR-rFVIIa | 1.6 | mg/ml |
| CaCl₂ | 10 | mM |
| L-Histidine | 10 | mM |
| Methionine | 1.0 | mg/ml |
| Tween 80 | 0.1 | mg/ml |
| pH 6.5 | | |

The solution was prepared from a purified bulk solution by buffer exchange on a gel filtration column. The solution was then sterile filtered, filled in sterile glass cartridges (1.6 ml/cartridge) closed with bromobutyl rubber plungers and laminate membranes, and stored at 5° C. and 30° C. Samples were analysed after storage for 0, 1, and 2 months. Contents of dimers, oligomers, and polymers were determined by GP-HPLC and contents of heavy chain fragments and oxidised forms were determined by RP-HPLC. The activity was determined by an amidolytic assay.

| | | 5° C. | 30° C. | |
|---|---|---|---|---|
| Parameter | 0 month | 1 month | 1 month | 2 months |
| pH | 6.54 | 6.70 | 6.70 | 6.68 |
| Visual inspection | Clear to | Clear to | Clear to | Clear to |
| | almost | almost | almost | almost |
| | clear | clear | clear | clear |
| Dimers/oligomers (%) | <0.3 | <0.3 | <0.3 | n.a. |
| Polymers (%) | <0.3 | <0.3 | <0.3 | n.a. |
| Total Protein (mg/mL) | 1.59 | 1.64 | 1.61 | 1.65 |
| Oxidised forms (%) | 1.9 | 1.8 | 2.3 | 2.4 |
| Heavy chain fragments (%) | 8.9 | 8.8 | 9.0 | 9.2 |
| Residual rFVIIa (%) | 0.15 | 0.17 | 0.28 | n.a. |
| Specific inhibitory amidolytic | 25.86 | 20.64 | 21.35 | 20.88 |
| activity (U/mg) | | | | |

n.a.: not analysed

Description of the Amidolytic Assay

An amidolytic assay is used to determine the inhibitory amidolytic activity of FFR-rFVIIa. The amidolytic assay is performed in microtiter plates and based on the following principle: FFR-rFVIIa and rFVIIa compete for the binding to Tissue Factor (TF) in a calcium-containing buffer for a fixed time period. The rFVIIa/TF complex, in contrast to the FFR-rFVIIa/TF complex, possesses a considerable amidolytic activity, which can be determined by cleavage of a chromogenic substrate. Cleavage of the chromogenic substrate results in release of the chromophore p-nitoanilin (pNA), which can be measured by absorbance at 405 nm using 620 nm as reference.

Therefore, for increasing amounts of FFR-rFVIIa added to a fixed amount of rFVIIa, a dose-dependent inhibition of amidolytic activity is observed. Depicting the measured absorbance versus the FFR-rFVIIa concentration in a log-log scale, a linear does-response curve will be obtained. The inhibitory amidolytic activity, relative to the in-house primary FFR-rFVIIa reference standard, is calculated using parallel-line statistics.

The specific inhibitory amidolytic activity is calculated by dividing the inhibitory amidolytic activity (U/mL) (analysis 434-1010) with the total protein content (mg(mL) (analysis 434-1011).

1. A liquid, aqueous composition, comprising
(i) a modified factor VII polypeptide;
(ii) an agent suitable for keeping pH in the range of from about 4.0 to about 8.0;
(iii) an antioxidant; and
(iv) an agent selected from the list of: a calcium salt, a magnesium salt, or a mixture thereof.

2. A composition according to claim 1, wherein the pH is kept in the range of from about 4.0 to about 7.0.

3. A composition according to claim claim 1, wherein the antioxidant (iii) is selected from the group consisting of: L- or D-methionine, a methionine analogue, a methionine-containing peptide, a methionine-homologue, ascorbic acid, cysteine, homocysteine, gluthatione, cystine, and cysstathionine.

4. A composition according to claim 3, wherein the antioxidant is L-methionine.

5. A composition according to claim 1, wherein the antioxidant is present in a concentration of from about 0.1 to about 5.0 mg/ml.

6. A composition according to claim 1, further comprising (v) a tonicity modifying agent.

7. A composition according to claim 6, wherein the tonicity modifying agent (v) is selected from the group consisting of: a neutral salt; a mono-, di- or polysaccharide; a sugar alcohol; an amino acid; or a small peptide, and a mixture of at least two of said modifying agents.

8. A composition according to claim 7, wherein tonicity modifier is mannitol and/or a neutral salt.

9. A composition according to claim 6, wherein the tonicity modifying agent (v) is present in a concentration of from 1 mM to 500 mM

10. A composition according to claim 9, wherein the concentration is 10-250 mM.

11. A composition according to claim 1, further comprising (vi) a non-ionic surfactant.

12. A composition according to claim 11, wherein the non-ionic surfactant is a polysorbate or a poloxamer or a polyoxyethylene alkyl ether.

13. A composition according to claim 1, wherein the agent (ii) suitable for keeping pH in the range of from about 4.0 to about 8.0 is selected from the group consisting of acids and salts of: citrate, acetate, histidine, malate, phosphate, tartaric acid, succinic acid, MES, HEPES, Imidazol, TRIS, lactate, glycylglycin, PIPES, glycin, and a mixture of at least two of said agents.

14. A composition according to claim 13, wherein the concentration of the agent (ii) is from about 1 mM to about 50 mM.

15. A composition according to claim 14, wherein the concentration of the agent (ii) is about 10 mM.

16. A composition according to claim 1, wherein the calcium and/or magnesium salt (agent (iv)) is present in a concentration of from about 5 mM to about 150 mM.

17. A composition according to claim 1, wherein the calcium salt is selected from the group consisting of: calcium chloride, calcium acetate, calcium gluconate, and calcium laevulate.

18. A composition according to claim 1, wherein the magnesium salt is selected from the group consisting of: magnesium chloride, magnesium acetate, magnesium sulphate, magnesium gluconate, and magnesium laevulate.

19. A composition according to claim 1, further comprising (vii) a preservative selected from the group consisting of phenol, benzyl alcohol, orto-cresol, meta-cresol, para-cresol, methyl paraben, propyl paraben, benzalconium chloride, and benzaethonium chloride.

20. A composition according to claim 1, wherein said composition is isotonic.

21. A composition according to claim 1, which is formulated for pharmaceutical administration.

22. A composition according to claim 1, wherein said modified Factor VII polypeptide is stable for at least 6 months at 2-8° C.

23. A composition according to claim 1, wherein the modified factor VII polypeptide has a biological activity relative to wild-type factor VIIa of less than about 25% of the specific activity of wild-type factor VIIa when tested in one or more of a clotting assay, proteolysis assay, or TF binding assay.

24. A composition according to claim 1, wherein the modified factor VII polypeptide is selected from the group consisting of: human and bovine factor VII, wherein the active site residue Ser344 is modified, replaced with Gly, Met, Thr, or Ala; human factor VII, wherein the residue Lys341 is replaced; human factor VII, wherein the residue Asp242 is replaced; human factor VII, wherein the residue His193 is replaced; FVII-(K341A); FVII-(S344A); FVII-(D242A); FVII-(H193A); a factor VII polypeptide modified in the active site by reaction with a reagent selected from the list of: peptide chloromethylketones or peptidyl cloromethanes; azapeptides; acylating agents such as various guanidinobenzoate derivatives and 3-alkoxy-4-chloroisocoumarins; sulphonyl fluorides such as phenylmethylsulphonylfluoride (PMSF); diisopropylfluorophosphate (DFP); tosylpropylchloromethyl ketone (TPCK); tosylysylchloromethyl ketone (TLCK); nitrophenylsulphonates; heterocyclic protease inhibitors such as isocoumarines, and coumarins; a factor VII polypeptide modified in the active site by reaction with a reagent selected from the list of: L-Phe-Phe-Arg chloromethyl ketone, D-Phe-Phe-Arg chloromethyl ketone, L-Phe-Pro-Arg chloromethyl ketone, D-Phe-Pro-Arg chloromethyl ketone, L-Glu-Gly-Arg chloromethyl ketone, D-Glu-Gly-Arg chloromethyl ketone, Dansyl-L-Phe-Phe-Arg chloromethyl ketone, Dansyl-D-Phe-Phe-Arg chloromethyl ketone, Dansyl-L-Phe-Pro-Arg chloromethyl ketone, Dansyl-D-Phe-Pro-Arg chloromethyl ketone, Dansyl-L-Glu-Gly-Arg chloromethylketone, and Dansyl-D-Glu-Gly-Arg chloromethylketone.

25. A composition according to claim 24, wherein the modified factor VII polypeptide is selected from the group consisting of: FVII-(S344A); FVII-(H193A); and a factor VII polypeptide modified in the active site by reaction with a reagent selected from the group consisting of: L-Phe-Phe-Arg chloromethyl ketone, D-Phe-Phe-Arg chloromethyl ketone, L-Phe-Pro-Arg chloromethyl ketone, D-Phe-Pro-Arg chloromethyl ketone, L-Glu-Gly-Arg chloromethyl ketone, D-Glu-Gly-Arg chloromethyl ketone, Dansyl-L-Phe-Phe-Arg chloromethyl ketone, Dansyl-D-Phe-Phe-Arg chloromethyl ketone, Dansyl-L-Phe-Pro-Arg chloromethyl ketone, Dansyl-D-Phe-Pro-Arg chloromethyl ketone, Dansyl-L-Glu-Gly-Arg chloromethylketone, and Dansyl-D-Glu-Gly-Arg chloromethylketone chloromethylketone, Dansyl-D-Phe-Pro-Arg chloromethylketone, Dansyl-L-Glu-Gly-Arg chloromethylketone, and Dansyl-D-Glu-Gly-Arg chloromethylketone.

26. A composition according to claim 1, wherein the modified factor VII polypeptide is present in a concentration of from about 0.1 mg/ml to about 15 mg/ml.

27. A method for preparing a liquid aqueous composition of a modified factor VII polypeptide, comprising providing a modified factor VII polypeptide in a solution comprising (ii) an agent suitable for keeping pH in the range of from about 4.0 to about 8.0; (iii) an antioxidant; and (iv) an agent selected from the list of: a calcium salt, a magnesium salt, or a mixture thereof.

28. A method for inhibiting blood clotting in a subject, the method comprising administering to a subject in need thereof an effective amount of an aqueous liquid composition comprising (i) a modified factor VII polypeptide, (ii) an agent suitable for keeping pH in the range of from about 4.0 to about 8.0; (iii) an antioxidant; and (iv) an agent selected from the list of: a calcium salt, a magnesium salt, or a mixture thereof.

29. A method for inhibiting tissue factor mediated reactions in a subject, the method comprising administering to a subject in need thereof an effective amount of an aqueous liquid composition comprising (i) a modified factor VII polypeptide, (ii) an agent suitable for keeping pH in the range of from about 4.0 to about 8.0; (iii) an antioxidant; and (iv) an agent selected from the list of: a calcium salt, a magnesium salt, or a mixture thereof.
